(19) United States
(12) Patent Application Publication
(10) Pub. No.: US 2011/0034761 A1
(43) Pub. Date: Feb. 10, 2011

(54) GASTRIC RING WITH MEMBRANOUS BRIDGE

(75) Inventor: Jean-Paul Ricol, Saint Germain sur I' Arbresle (FR)

Correspondence Address: OCCHIUTI ROHLICEK & TSAO, LLP, 10 FAWCETT STREET, CAMBRIDGE, MA, 02138, US

(73) Assignee: Compagnie Europeene d'Etude et de Recherche de Dispositifs pour I'lmplantation par Laparoscopie, Vienne (FR)

(21) Appl. No.: 12/937,059

(22) PCT Filed: Apr. 10, 2009
(86) PCT No.: PCT/FR09/50669

§ 371 Date: Oct. 8, 2010

(30) Foreign Application Priority Data
Apr. 14, 2008 (FR) 08 02030

(51) Int. Cl. A61F 2/04

(52) U.S. Cl. 600/37

(57) ABSTRACT

The invention relates to an implantable surgical ring (1) intended to close on itself around a mean extension axis in order to enable adjustment of the section through which a biological organ passes, said ring being characterized in that the same comprises at least one first and one second gripping bag (10, 11) that are tiered along the mean extension axis, are designed to be filled with filling fluid, and respectively have a first peak portion (12) and a second peak portion (13), and in that said first and second peak portions (12, 13) are separated by a separation area (14) including a retaining means (20) receding away from said first and second peak portions (12, 13) to limit the depth (P) to which the gripped biological organ can enter between said first and second peak portion.

### TECHNICAL FIELD

The present invention pertains to the general technical field of surgical implants designed to be implanted in a patient's body around biological organs constituting a pocket or a duct and more particularly gastric rings designed to form a closed loop around the stomach in order to reduce the diameter of the opening of the stoma.

The present invention pertains more particularly to an implantable surgical ring designed to be closed on itself in order to form a closed loop around a biological organ constituting a pocket or a duct in order to modify the section of passage of said biological organ.

### PRIOR ART

There are known ways of practicing surgical operations on patients suffering from severe obesity who, because of their excess weight, are exposed not only to physical discomfort but also to psychological burdens as well as ancillary illnesses such as diabetes, cardiovascular illnesses or even severe arthritis.

In particular, a prior art technique consists in making a gastric constriction to reduce the size of the stomach and therefore the consumption of food.

To this end, it is common practice to resort to the use of gastroplasty rings implanted around the patient's stomach in order to reduce its volume as well as the diameter of its passage (namely the stoma).

It is well known that such gastroplasty rings generally comprise a flexible band made of elastomer material designed to be closed at both ends by appropriate closing means in order to grip the stomach.

Furthermore, known rings generally comprise a ring-shaped compression chamber situated on the internal face of the flexible band, with a volume that can be adjusted by the addition or removal of a filler fluid.

It is thus possible, using a ring with fixed overall dimensions, to finely adjust the internal diameter of said ring by radial expansion or retraction of the chamber.

Such implantable rings are generally satisfactory but suffer however from a certain number of drawbacks, especially due to uncontrolled changes in position during the treatment.

Indeed, it turns out to be the case that the prior art rings sometimes undergo shifts prompted by the natural movements of expansion and contraction of the stomach.

In certain cases, these movements may lead to the expulsion of the ring by slippage or again the ring may be overturned on itself in such a way that it becomes therapeutically inoperative and a source of discomfort for the patient.

In such a situation, it therefore becomes necessary to carry out a further surgical operation in order to replace or reposition the accidentally shifted ring.

Naturally, the repetition of these corrective surgical operations unnecessarily mobilizes medical staff, causes additional unpleasantness to the patient and even exposes him or her to post-operational complications.

Furthermore, the prior-art rings are sometimes liable to cause deterioration to the stomach wall, such as injuries by abrasion or even necrosis by pinching, especially when the ring is badly positioned and/or when the compression chamber has pads of material or folds which locally trap said stomach wall.

### SUMMARY OF THE INVENTION

The objects assigned to the present invention are therefore aimed at remedying the above-mentioned drawbacks and proposing a novel implantable surgical ring, especially a gastric ring, which has great stability while particularlyly respecting the integrity of the living tissues on which it acts.

Another object assigned to the present invention is aimed at proposing a novel implantable surgical ring having a simple, compact, light and robust structure.

Another object assigned to the present invention is aimed at proposing a novel implantable surgical ring which is particularly comfortable and has a great faculty of adapting to different conditions of use.

The objects assigned to the invention are achieved by means of an implantable surgical ring designed to be closed on itself in order to form a closed loop around a mean axis of extension (X-X′) so that it can grip a biological organ constituting a pocket or a duct in order to modify the section of passage of said biological organ, said ring being characterized in that it comprises at least one first gripping pocket and one second gripping pocket which are tiered relative to each other along the mean axis of extension (X-X′) and designed to be filled with a filler fluid so as to provide for the gripping of the biological organ, said first and second gripping pockets respectively having a first peak portion and a second peak portion which project towards the interior of the loop to push against said biological organ, and wherein said first and second peak portions are separated from each other by a separation zone which defines a matter-free clearance zone sufficiently marked to provide for a discontinuity of contact between the biological organ and the surgical ring, between the peak portions, and comprising a retaining means receding away from said first and second peak portions and designed to limit the depth at which the gripped biological organ is liable to penetrate between said first and second peak portions so as to prevent damage to the wall of the biological organ in the separation zone.

### BRIEF DESCRIPTION OF THE DRAWINGS

Other objects, features and advantages of the invention shall appear in greater detail from the following description as well as from the appended drawings, given purely on an illustrative and non-restrictive basis, of which:

FIG. 1 is a view in perspective of a surgical ring according to the invention in its closed configuration.

FIG. 2 is a cutaway view in perspective of a portion of the surgical ring shown in FIG. 1.

FIG. 3 illustrates a longitudinal view in perspective of a surgical ring according to the invention placed in a closed configuration around a biological organ in order to grip this organ.

FIGS. 4A, 4B and 4C provide an illustration, along partial schematic views in longitudinal section, of different alternative embodiments of surgical rings according to the invention.

### BEST EMBODIMENT OF THE INVENTION

The present invention pertains to an implantable surgical ring 1 which is designed to be closed on itself in order to form a closed loop 2 around a mean axis of extension (X-X′) so as to capable of gripping a biological organ 3 which is a pocket or a duct in order to modify the section of passage of said biological organ 3.

In other words, the loop 2 is geometrically wound about a mean axis of extension (X-X′), which advantageously coincides, after implantation, with the direction of extension of the duct or pocket formed by the biological organ 3, as shown in FIG. 3.

In the following description, reference is made by way of an example and for convenience of description, to a gastroplasty ring or gastric ring designed to be implanted around the stomach in order to reduce the diameter of the opening of the stoma or designed to be implanted around the esophagus.

However, the invention is in no way limited to this application and generally covers surgical rings designed to be implanted in the body of a patient around any biological organ whatsoever constituting a pocket or a duct in order to modify the section of passage of said biological organ 3 when said ring is positioned around this organ.

Thus, the present invention can be adapted to the treatment of urinary or fecal incontinence or again for the regulation of the blood flow.

Depending on its destination, the surgical ring can naturally be adapted to the dimensions, the environment and the sensitivity of the biological organ 3 concerned by constriction, such as the bladder, the urethra, the intestine, arteries, veins etc.

Preferably, the surgical ring 1 is flexible enough to pass from an open configuration (not shown) to a closed configuration illustrated especially in FIGS. 1 and 3, in which said ring is appreciably closed towards both ends 4, 5 by means of appropriate closing means 6, 7 so as to form the closed loop 2 mentioned here above.

Thus, by trapping the biological organ 3 within the loop 2, it is possible, by contraction, to reduce the diameter of its section of passage and, in the particular case of the stomach, the opening of the stoma.

Naturally, the geometry adopted by the closed loop 2 is in no way restricted. However this geometry will preferably show an appreciably rounded contour and in a particularly preferable way, an appreciably circular contour.

More generally, the surgical ring 1 of the invention preferably, when in a closed configuration, will appreciably have a geometry generated by an axis of revolution (X-X′).

Advantageously, the closing means 6, 7 are designed to cooperate with each other so as to lock the ring 1 in its closed configuration.

For example, to this end it is possible to use a sleeve 6 fixed to the first end 4 of the ring 1 and designed to receive a rod 7 associated with the second end 5, said rod 7 preferably comprising one or more snugs 7A so as to enable a clip-on type assembly with the sleeve 6.

According to one major characteristic of the invention, the surgical ring 1 has at least one gripping pocket 10 and one second gripping pocket 11 which are tiered relatively to each other along the mean axis of extension (X-X′) and are designed to be filled with a filler fluid so as to ensure the gripping of the biological organ 3.

To this end, the first gripping pocket 10 has a first peak portion 12 and the second gripping pocket 11 respectively has a second peak portion 13, said first and second peak portions projecting towards the interior of the loop 2 so as to press against the biological organ 3.

According to another major characteristic of the invention, said first and second peak portions 12 and 13 are separated from each other by a separation zone 14.

Advantageously, this separation zone 14 provides the ring 1 with a particularly stable nature once it is implanted.

Indeed, said separation zone enables the separation, in normal operation, of the first contact zone 16 where the first peak portion 12 comes into contact with the biological organ 3, from the second contact zone 17 where the second peak portion 13 presses against said biological organ 3.

Thus, by keeping two contact zones 16, 17 in an appreciably separated state, at a distance from each other, the separation zone 14 provides for discontinuity of the anchoring of the ring 1 to the organ 3, thus significantly improving the stability of said ring inasmuch as, even if said ring momentarily loses its hold at the first anchoring point given to it by the first gripping pocket 10, there remains the second anchoring point, given to it by the second gripping pocket 11 in order to retain it, and vice versa.

In other words, the separation of the gripping means increases the holding points of the ring 1 to the organ 3 and thus considerably reduces the probability that kinematic conditions of a shifting of said ring relative to said biological organ will all occur at the same point in time.

Consequently, the phenomena of shifting or even the accidental release of the ring are appreciably limited.

As understood in the invention, the first gripping pocket 10 and the second gripping pocket 11 can be walled off and separated from each other imperviously or on the contrary they may communicate with each other and form two projecting portions of a same chamber.

According to an alternative embodiment of this kind, the chamber forming a common container for the filler fluid has two distinct and distant outgrowths which constitute the first and second peak portions 12, 13 and are designed to each come into contact with the biological organ 3 respectively at the level of the first contact zone 16 and the second contact zone 17.

Furthermore, in a particularly preferable manner, each gripping pocket 10, 11 and more particularly each of the first and second peak portions 12, 13 is appreciably ring-shaped and extends appreciably over the entire perimeter of the loop 2.

Thus, the surgical ring 1 according to the invention preferably has two ring-shaped gripping pockets axially superimposed and distant from each other, each one of them being capable of providing for the constriction of a distinct portion of the biological organ 3, in this case at the level of a first and second contact zone 16, 17 situated at different x-axis points along the axis of extension (X-X′) and having an appreciably circular contour centered on said extension axis (X-X′).

A tiering of peak portions of this kind and more generally of gripping pockets as a whole advantageously provides for a firm and secure holding of the ring on the biological organ 3 and especially maintains said ring in position during the peristaltic motions of said biological organ 3.

In order to mark the transition between the first and second gripping pockets 10, 11 and more particularly between the first peak portion 12 and the second peak portion 13, the separation zone 14 preferably defines a material-free clearance zone that forms a marked depression 18 that is hollow relative to said peak portions 12, 13 and opens into interior of the loop 2.

More particularly, the bottom of said depression 18 can appreciably follow the shape of a circular contour about the axis (X-X′) whose diameter is greater than that of the contour formed by circular peak lines corresponding to the first and second gripping pockets 10, 11.

Thus, once the ring has been implanted, the separation zone 14 advantageously faces the wall of the biological organ 3 and forms a mechanical isolating boundary between the first contact zone 16 and the second contact zone 17, i.e. a zone in which the contact between the external surface of the biological organ 3 and the internal surface of the ring 1 is normally interrupted, at least in places, in such a way that said surgical ring 1 behaves independently in each of said contact zones 16, 17.

In particular, the invention advantageously prevents a slippage of the ring 1 through a breaking of contact in the first contact zone 16 from causing a total slippage of the ring by a continuous prolongation of the conditions in which said dynamic phenomenon of slippage arises in the second contact zone 17.

By analogy, the structure of the surgical ring 1 according to the invention recalls that of a catamaran whose floaters correspond to the gripping pockets 10, 11 which mainly in a simultaneous manner and very occasionally in an individual manner provide for contact with the surface of the biological organ 3.

Naturally, the layout and shape of the first and second gripping pockets 10, 11 which appreciably define, by construction, the geometry of the first and second contact zones 16, 17, are not limited to one particular alternative embodiment.

Owing to the presence and shape of the separation zone 14 according to the invention, the wall of the biological organ 3 is capable, when the ring 1 is implanted, of mouving about in the space thus created between the peak portions 12, 13 and especially of creeping in between the first and second gripping pockets 10, 11. In certain conditions, for example when food is passing through, as in the case of the stomach, the wall of the organ may temporarily have a tendency to engage with a certain penetrative force into the separation zone 14 and sinking into it in depth.

This is why, according to another major characteristic of the invention, the separation zone 14 has a retaining means 20 positioned in a receding position from the first and second peak portions 12, 13 and designed to limit the depth P at which the clamped biological organ 3 is liable to penetrate between said first and second peak portions 12, 13 so as to prevent any deterioration in the wall of the biological organ 3 in the separation zone 14.

Thus, if the separation zone 14 is sufficiently marked to provide for a certain discontinuity, during the normal operation of the device, of the contact between the biological organ 3 and the surgical ring 1, between the peak portions 12, 13, it is nevertheless provided with a retaining means 20 which prevents said biological organ 3 from penetrating, even temporarily or accidentally, too deeply between the gripping pockets 10, 11 and, to this end, advantageously limits the free space made to enable the free radial movement of the wall of the organ.

Indeed, an excessive penetration would lead to the formation of a marked fold of the wall of the biological organ and would entail a risk of gradual damage to the tissues of said biological organ 3 by pinching, abrasion or necrosis.

Advantageously, the retaining means 20 therefore forms a stop against the progress of the biological organ 3 between the structures of the ring in the separation zone 14 when said organ tends to locally follow an appreciably radial motion of expansion oriented toward the exterior of the loop 2.

As it happens, the limit of penetration defined by the retaining means 20 is situated at a depth P relative to the peak portions 12, 13.

Geometrically, when the loop 2 is appreciably circular, the depth P may be considered to be the difference between the base radius of a first fictitious cylinder C1 having the axis (X-X′) and being inscribed in the loop i.e. tangential to the first and second peak portions, and the base radius of a second fictitious cylinder C2 having the same axis (X-X′) and being tangential to the face of the retaining means 20 most distant from the axis (X-X′), i.e. more particularly tangential to the bottom of the depression 18 as illustrated in FIG. 3.

More particularly, the retaining means 20 is arranged relative to the gripping pockets 10, 11 in such a way that the radius of curvature of the wall of the biological organ 3, even assuming that said wall comes into contact with the support surface borne by said retaining means, is permanently held at a value appreciably greater than or equal to a critical value below which there is a risk of abrasion and necrosis of said wall.

In other words, the retaining means 20 and more generally the separation zone 14 are arranged to provide for a certain continuity of the profile of the wall of the biological organ 3, preventing the formation of marked folds or of compression in order to preserve said biological wall within and in the vicinity of said separation zone 14, especially within the depression 18 and more generally throughout the space between the peak portions 12, 13.

Thus, while advantageously preserving the robustness and stability of an anchoring comprising at least two independent pressing points, the surgical ring 1 of the invention is particularly non-traumatic since it prevents any bruising of the biological organ related to the implementation of the particular gripping structure.

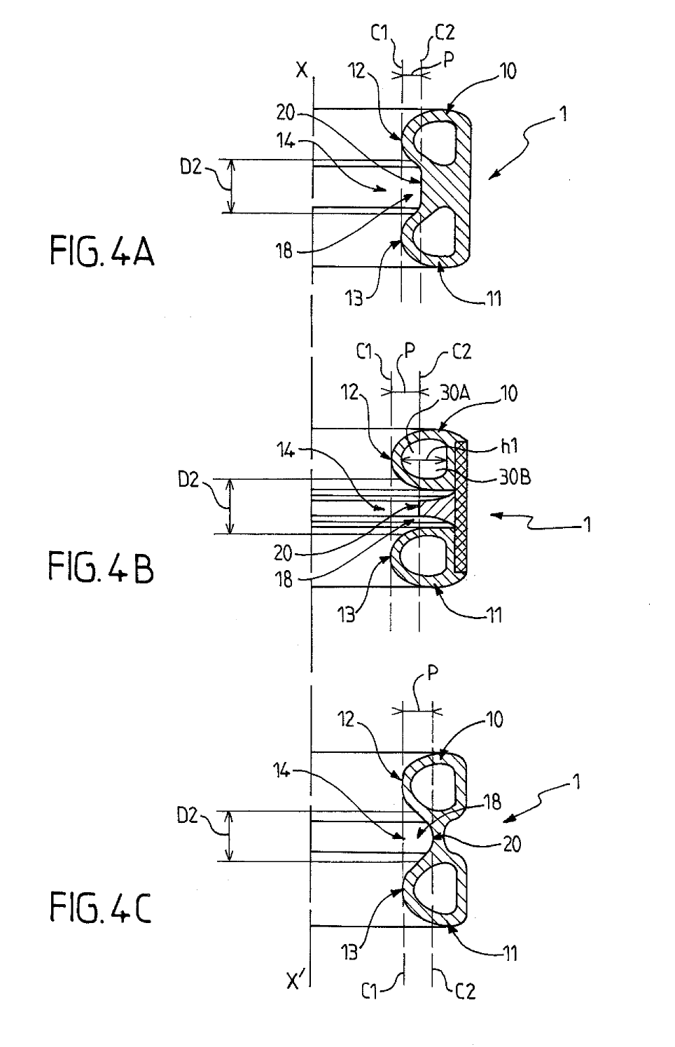

According to one embodiment, the retaining means 20 may be separated from the first and/or second gripping pocket 10, 11 and especially can be disjoined from the walls demarcating said gripping pockets, as illustrated in FIG. 4B.

In particular, the retaining means 20 can be formed by a crenellated structure or a bead-like element occupying the space between the gripping pockets 10, 11, culminating in the receding of the peak portions 12, 13 and separated from the walls of the neighboring gripping pockets 10, 11, by residual interstices that are too narrow to permit the penetration of the wall of the biological organ 3.

However, the retaining means 20 preferably connects the first gripping packet 10 to the second gripping packet 11 as illustrated especially in FIGS. 1 to 3, 4A and 4C.

Thus, the retaining means 20 could form a means of continuous junction between said gripping pockets 10, 11 and create a direct mechanical link between these gripping pockets.

In a particularly preferred way, the retaining element 20 forms one piece with the first gripping pocket 10 and/or with the second gripping pocket 11.

Advantageously, a structural unity of this kind of the ring 1 gives the ring a particularly robust, compact and light structure that is easy to manufacture, especially for particularly simplified alternative embodiments such as those illustrated in FIGS. 4A and 4C.

Furthermore, the surgical ring 1 according to the invention preferably comprises a dorsal belt 21 supporting the first and second gripping pockets 10, 11.

Naturally, said dorsal belt 21 is flexible enough to permit the opening and closing of the ring and to be capable of being wound on itself so as to form the loop 2 and at the same time so as to be sufficiently resistant to deformation and especially to tensile forces to serve as a stable pressing point for the gripping pockets 10, 11.

Preferably, once the ring has been locked by means of its closing means 6, 7, the dorsal belt 21 is non-extensible enough to provide an appreciably constant perimeter, whatever the constraints exerted by the biological organ 3 on the gripping pockets 10, 11 in such a way that it provides a radial support to these pockets.

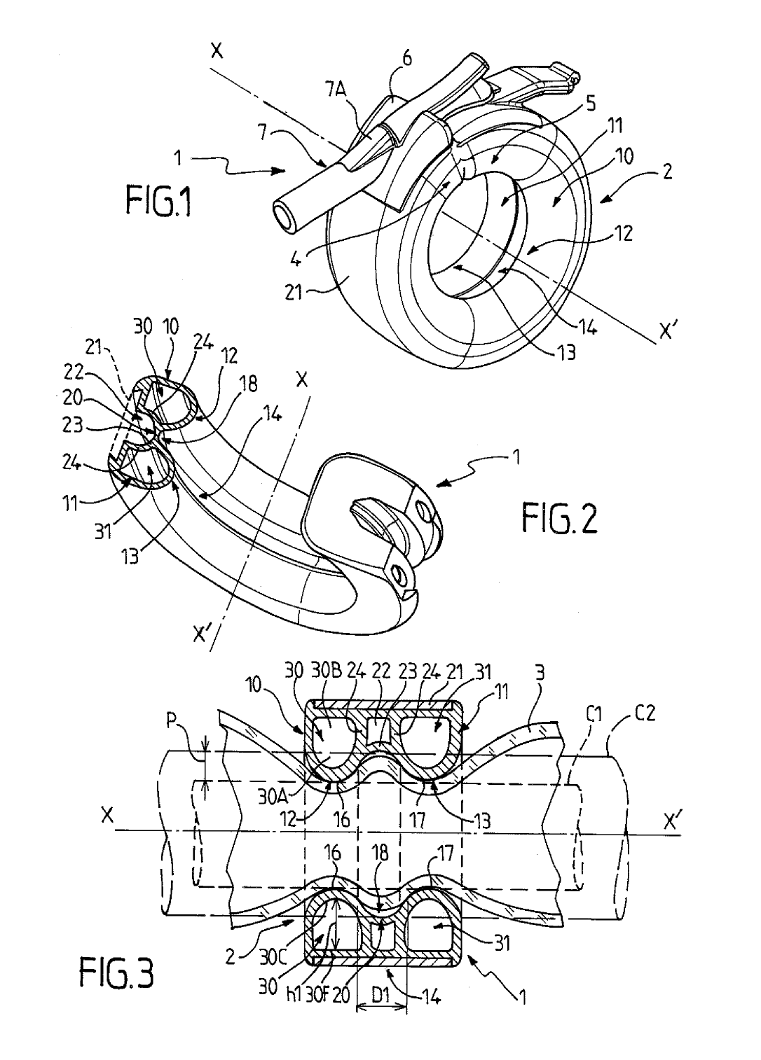

As illustrated in FIGS. 2, 3 and 4B, the dorsal belt 21 may take the form of a band with an appreciably rectangular section on the internal faces of which said first and second gripping pockets 10, 11 are fixed.

Advantageously, the surgical ring 1 of the invention may be laid out in such a way that the first and/or second gripping pocket 10, 11 have a deformable structure that can permit the subsidence of the first peak portion 12 and/or respectively of the second peak portion 13 towards the exterior of the loop 2, under the constraint of expansion of the biological organ 3.

In particular, the first and/or second gripping pocket 10 or 11 may to this end be made out of a flexible material and especially out of biocompatible polymers such as silicone.

Thus, when the first and second gripping pockets 10, 11 are filled below their nominal volume and/or below their nominal pressure, they have a certain flexibility and may consequently get deformed and especially crushed in response to the constraint exerted on them by the biological organ 3 when it tends to expand, especially during natural peristaltic movements associated with digestion.

In other words, the first and second gripping pocket 10, 11, preferably have a functional flexibility which, as the case may be, can be controlled by the conditions of filling of said pockets.

The term “nominal inflation volume” designates a quantity of fluid which may be contained in the first gripping pocket 10 and the second gripping pocket 11 respectively without the wall that demarcates it undergoing an elastic extension or even a distension under the effect of the inflation.

To this end, it is noteworthy that the first and second gripping pocket 10, 11 can equally well, as understood in the invention, have either a semi-rigid character, i.e. in being preformed to take a shape at rest, even when there is no inflation fluid, corresponding to the shape they had when they contained a nominal volume of said inflation fluid, or a “sagging character”, i.e. having no shape of their own nor occupying a particular deployed volume so long as they are not filled with said filler fluid.

Preferably, the retaining means 20 also has a structure that is sufficiently deformable to appreciably go along with the subsidence of said first and second peak portions 12, 13 towards the exterior of the loop 2.

In other words, while said retaining means 20 is designed to be an obstacle to the penetration of the biological organ 3 between said first and second peak portions 12, 13, on the contrary it does not form a projecting residual pressing point capable of stressing the wall of the biological organ 3 and more particularly of exerting constriction on said biological organ 3 that is more pronounced than the constriction exerted by the peak portions 12, 13 when these portions recede in moving the axis (X-X′) away under the pressure exerted by said biological organ 3.

Thus, the surgical ring 1 is liable to pass from a “narrowed” configuration in which the gripping pockets 10, 11 are appreciably inflated to their nominal volume and completely straightened towards the interior of the loop to a “widened” configuration in which said gripping pockets 10, 11 and especially the first and second peak portions 12, 13 have appreciably subsided, i.e. are at a greater distance from the axis (X-X′) than in a narrowed configuration.

Naturally, the passage from the narrowed configuration to the widened configuration, which can be obtained by under-inflation, especially by draining filler fluid when it is incompressible (in the case of physiological serum) or by simple elastic effect when said fluid is compressible (as in the case of a gas) results in an increase in the diameter of constriction of the biological organ 3.

Advantageously, the functional flexibility of the retaining means 20 makes it possible, even in a widened configuration, to preserve a residual depth of clearance in the separation zone 14, i.e. to preserve an appreciably concave profile of the ring relative to the biological organ 3 between the first and second peak portions 12, 13.

Although the retaining means may show a compressible solid structure, which partially fills the separation zone 14 contiguously or interruptedly as illustrated in FIGS. 4A and 4B, the ring 1 of the invention preferably has a recess 22 made on the external side of the loop 2 relative to the retaining means 20, between the first and second gripping pockets 10, 11 and perpendicular to said retaining means 20 so as to facilitate the subsidence of these means as illustrated especially in FIGS. 2 and 3.

More particularly, said recess 22 corresponds to a compressible cavity demarcated by the retaining means 20, the side walls of the first and second gripping pockets 10, 11, situated facing one another on either side of said retaining means 20 and the dorsal belt 21, as illustrated in FIGS. 2 and 3.

Advantageously, a recess 22 of this kind forms a sort of cushion which can be deformed by compression, liable to get crushed in a centrifugal radial direction and acting as a shock absorber with a certain radial “reserve of deformation”.

Furthermore, the dorsal belt 21 preferably has one or more holes (not shown) that put the compressible cavity into communication with the exterior of the ring, so as to permit variations in volume of said cavity under the effect of the deformations induced by the radial movements of the peak portions 12, 13 and of the retaining means 20.

According to a preferable embodiment, as illustrated in FIGS. 2 and 3, the retaining means 20 comprise a bridge provided with an apron 23 designed to form a stop against the biological organ 3, said apron 23 being supported by one or more flexible pillars 24 which take support on the dorsal belt 21 and are designed to permit the subsidence of said apron 23 into the exterior of the loop 2.

According to one preferred embodiment, the pillars 24 blend with the side portions of the walls which demarcate the first and second gripping pockets 10, 11 and are located so as to be facing each other on either side of the retaining means 20.

Thus, the retaining means 20 can be formed by a bridge, the apron 23 of which connects the first gripping pocket 10 to the second gripping pocket 11 so that said bridge straddles the recess 22. Advantageously, the fastening points of the apron 23 are positioned so as to be receding from the corresponding first and second peak portions 12, 13.

Preferably, the bridge thus sets up a junction between the first and second gripping pockets throughout the length of said pockets, i.e. appreciably throughout the perimeter of the loop 2.

Whatever the embodiment considered, the retaining means 20 is preferably made out of a biocompatible elastomer material such as silicone.

In particular, it is possible to use a silicone membrane with a Shore hardness appreciably equal to 30 and having a thickness appreciably ranging between 0.5 and 0.6 mm to make the apron 23.

Advantageously, said membrane may form one piece with the wall of either of the gripping pockets 10, 11, thus simplifying the manufacture of the ring and improving the robustness of the unit.

Besides, the retaining means 20 is preferably arranged so that when the first gripping pocket 10 is filled with filler fluid and more particularly contains a quantity of fluid corresponding to its nominal filling volume, the volume occupied by said filler fluid within said first gripping pocket extends in a direction transversal to the axis (X-X′) on either side of the retaining means 20 and more specifically on either side of the radial limit of penetration that the retaining means 20 defines counter to the biological organ 3.

In other words, the fictitious cylinder C2 geometrically subdivides the first cavity 30 demarcated by the first gripping pocket 10 into two “sub-cavities” 30A, 30B both containing filler fluid, in this case an upper sub-cavity 30A which extends between the first peak portion 12 and the retaining means 20 and a lower sub-cavity 30B which extends between the retaining means 20 and the dorsal belt 21 of the ring.

Advantageously, the ring 1 of the invention therefore possesses a dual hydraulic and/or pneumatic radial suspension and damping system that is both above and below the bridge, giving it excellent functional flexibility both at the peak portions 12, 13 and at the retaining means 20.

As understood in the invention, the first cavity 30 designed to contain the filler fluid preferably extends between a bottom wall 30F oriented toward the exterior of the loop 2 and a peak wall 30C oriented toward the interior of the loop and situated at a distance h₁ from said bottom wall 30F.

Thus, the first cavity 30 has a useful height h₁ measured between said bottom wall 30F and the peak wall 30C.

Preferably, the bottom wall 30F is situated in the vicinity of the dorsal belt 21 while the peak wall 30C is immediately below the peak portion 12 and forms the surface of the membrane that demarcates the first gripping pocket 10 in said first peak portion 12.

Preferably, the retaining means 20 is situated at a level between 40% and 60% of said useful height h₁ relative to the peak wall 30C, especially when the first cavity 30 is filled to its nominal volume.

Naturally, all the constructional arrangements pertaining to the first gripping pocket 10 and the first cavity 30 preferably apply proportionately to the second cavity 31 demarcated by the second gripping pocket 11.

In a particularly preferred manner, the first and second gripping pockets are appreciably images of one another relative to a median plane of symmetry normal to the axis (X-X′) and intersecting the ring, and especially the retaining means 20 and/or the dorsal belt 21 at its middle.

Furthermore, the retaining means 20, the first gripping pocket 10 and the second gripping pocket 11 are preferably laid out so that, between the first and second peak portions 12, 13, they demarcate a depression 18 for which the ratio R1 of the width at mid-depth D1 to the depth P is greater than or equal 0.5 (i.e 50%) and preferably appreciably included between 0.5 and 1 (i.e. between 50% and 100%).

In other words we have:

$$0 , 5 \leq R 1 = \frac{D 1}{P} \leq 1$$

where P represents the maximum depth of penetration of the biological organ 3 as demarcated by the retaining means 20, when the gripping packets are filled to their nominal volume and under normal conditions of operation, and where D1 represents the width of the separation zone 14, i.e. the distance between the side walls of said gripping pockets 10, 11 measured at the respective intersections of said gripping pockets with a third fictitious cylinder having the axis (X-X′) and a radius equal to the half-sum of the radii of the first and second fictitious cylinders C1, C2 as illustrated in FIGS. 3, 4A, 4B and 4C.

According to a preferred alternative embodiment, the width at mid depth of said depression corresponds appreciably to the width of the recess 22 made beneath the bridge forming the retaining means 20.

Furthermore, the above-mentioned depression preferably has a ratio R2 of its width D2 at maximum penetration depth P to said maximum penetration depth P greater than or equally to 0.35 and preferably appreciably ranging from 0.35 to 1, i.e.:

$$0 , 35 \leq R 2 = \frac{D 2}{P} \leq 1$$

Preferably, the width D2 at maximum penetration depth corresponds appreciably to the minimum distance between the first gripping pocket 1 and the second gripping pocket 2 at the support surface of the retaining means 20, i.e. at the bottom (in this case an appreciably a flat bottom) of the depression 18 accessible to the biological organ 3.

Additionally, the ratio R1 is preferably greater than the ratio R2 i.e. the depression 18 of the separation zone 14 is concave and flares out with respect to the interior of the loop 2.

Advantageously, the sizing of said depression 18 furthermore ensures mechanical discontinuity in normal operation of the first and second contact zones 16, 17 and thus improves the mechanical stability of the ring on the biological organ 3 while avoiding the pinching of the wall of said biological organ 3 in a separation zone that might be too narrow.

Preferably, the above-mentioned proportions R1, R2 of the depression 18 correspond to the values considered in a nominal situation of operation of the ring 1. If necessary, they remain preferably appreciably included in the ranges indicated whatever the level of functional inflation of the gripping pockets 10, 11.

Thus, the implantable ring 1 of the invention gives a constriction that is reliable, comfortable and non-traumatic for the biological organ 3 to be gripped.

Naturally, it is possible to envisage adapting a retaining means according to any one of the variants described here above to a surgical ring whose gripping pockets are not tiered or not uniquely tiered along the axis of said ring, but situated appreciably at the same x-axis position along the axis (X-X′) so that each of them covers a predetermined angular sector about said axis (X-X′) in thus forming angular subdivisions of a same crown.

According to such an alternative embodiment, the separation zone 14 could comprise a trench whose walls extend appreciably in planes parallel to the axis (X-X′).

The working of one alternative embodiment of a gastric ring 1 according to the invention shall now be described briefly with reference to FIG. 3.

The ring is first of all introduced in an unrolled (or twisted) shape preferably by endoscopy.

It is then passed around the stomach 3 and then closed on itself and locked by means of closing means 6, 7 so as to form a closed loop 2.

The practitioner then introduces a determined quantity of filler fluid into the first and second gripping pockets 10, 11 in such a way that the first and second peak portions 12, 13 respectively lean on or firm up the support that they take on the stomach 3 at two disjoined contact zones 16, 17 separated by the intermediate separation zone 14.

Advantageously, the filling of the ring-shaped gripping pockets 10, 11 exerts a appreciably smooth, adjustable, centripetal radial pressure, distributed throughout the circular rim of each contact zone 16, 17, on the stomach 3 permitting a fine and personalized adjustment of the constriction of said stomach 3.

Once the ring has been implanted, and when there is no motion of the stomach 3, especially when the stomach is contracted, the intermediate portion of the ring wall included between the first and second contact zones 16, 17, moves freely in the space made at the separation zone 14.

Advantageously, not only does the invention prevent any deterioration of the tissues related to the friction of said intermediate portion against the ring 1 but it furthermore provides for a dual anchoring of said ring that is particularly stable.

When the stomach 3 tends to expand radially, for example when absorbing food, its expansion forces the stomach wall to slip in between the gripping pockets 10, 11, sinking in more deeply into the separation zone 14.

However, this penetration motion is limited by the retaining means 20 which stops the centrifugal radial progress of the stomach wall when said stomach wall abut the retaining means 20, i.e. when the stomach wall reaches the predetermined maximum depth of penetration P as illustrated in FIG. 3.

Thus, even if the stomach wall, under the pressure of the contents of the stomach which push it against the interior of the ring, temporarily and appreciably matches the concave contour of the depression 18, said stomach wall locally maintains a radius of curvature that is big enough not to get crushed in forming one or more tightly close folds.

If the ring is preformed, i.e. if the gripping pockets 10, 11 are arranged so that they themselves stay appreciably in a deployed position when they are at rest, and when the ring is under-inflated, i.e. when one of the pockets contains a quantity of filler fluid that is smaller than their nominal receiving capacity, then the radial expansion of the stomach 3 has the effect of pushing back the peak portions 12, 13 towards the periphery of the ring and thus tends to crush each gripping pocket on itself, according to an appreciably centripetal radial movement.

Advantageously, this flattening is distributed upstream to the retaining means 20, i.e. above the apron 23 of the bridge when the domes forming the projecting parts of the gripping pockets get deformed elastically and downstream of the retaining means, i.e. between the apron 23 and the dorsal belt 21, when the pillars 24 sag in crushing the compressible cavity 22.

In other words, the gripping pockets 10, 11 and the retaining means 20 subside jointly, in such a way that even if the radial distance between the peak line and the retaining means 20 is liable to diminish, these retaining means remain appreciably receded relative to the peak portions 12, 13 i.e. the profile of the separation zone advantageously keeps a certain functional concavity during the subsidence.

Furthermore, this controlled differential deformation gives the ring a high capacity of adaptation and of absorbing variations in diameter of the organ 3 related to the working of this organ.

Advantageously, when the stomach then returns to a retracted position, its wall is freely drawn back in a centripetal movement to the axis (X-X′) and does not remain wedged between the gripping pockets 10, 11.

More particularly, the intermediate portion of the stomach wall may get detached from the retaining means 20 and move away from it to return to its floating position without any risk of pinching or flattening.

Advantageously, the peak portions 12, 13 and the retaining means 20 make a concomitant elastic return when the stomach contracts while preserving both the independent double anchoring and the definition of a boundary depth of penetration P.

The behavior of the ring is thus particularly flexible, predictable and reproducible, which gives it great versatility and excellent reliability.

### BEST MANNER OF ACHIEVING THE INVENTION

The invention finds industrial application in the designing and manufacture of surgical rings and especially gastric rings.

1. Implantable surgical ring designed to be closed on itself in order to form a closed loop around a mean axis of extension so that it can grip a biological organ constituting a pocket or a duct in order to modify the section of passage of said biological organ, said ring comprising at least one first gripping pocket and one second gripping pocket which are tiered relative to each other along the mean axis of extension and designed to be filled with a filler fluid so as to provide for the gripping of the biological organ, said first and second gripping pockets respectively having a first peak portion and a second peak portion which project towards the interior of the loop to push against said biological organ, and said first and second peak portions are separated from each other by a separation zone which defines a matter-free clearance zone sufficiently marked to provide for a discontinuity of contact between the biological organ and the surgical ring, between the peak portions, and which comprises a retaining means receding away from said first and second peak portions and designed to limit the penetration depth at which the gripped biological organ is liable to penetrate between said first and second peak portions so as to prevent damage to the wall of the biological organ in the separation zone.

2. Surgical ring according to claim 1, wherein the retaining means, the first gripping pocket and the second gripping pocket demarcate a depression between the first and second peak portions, for which the ratio of the width at mid-depth to the penetration depth is greater than or equal 0.5 and preferably appreciably included between 0.5 and 1.

3. Surgical ring according to claim 1, further including a dorsal belt supporting the first and second gripping pockets.

4. Surgical ring according to claim 3, wherein the retaining means comprises a bridge provided with an apron designed to form a stop against the biological organ, said apron being supported by one or more flexible pillars which take support on the dorsal belt and are designed to permit the subsidence of said apron into the exterior of the loop.

5. Surgical ring according to claim 4, wherein the pillars are blended with the side portions of the walls which demarcate the first and second gripping pockets and which are located so as to be facing each other on either side of the retaining means.

6. Surgical ring according to claim 1, wherein the retaining means connects the first gripping pocket to the second gripping pocket.

7. Surgical ring according to claim 1, wherein the first and/or second gripping pocket have a deformable structure that can permit the subsidence of the first and/or second peak portions towards the exterior of the loop, under the constraint of expansion of the biological organ, and in that the retaining means also has a structure that is sufficiently deformable to appreciably go along with said subsidence.

8. Surgical ring according to claim 7, wherein it has a recess made on the external side of the loop relative to the retaining means, between the first and second gripping pockets and perpendicular to said retaining means so as to facilitate their subsidence.

9. Surgical ring according to claim 1, wherein the retaining means is preferably arranged so that, when the first gripping pocket is filled with filler fluid, the volume occupied by said filler fluid within it extends on either side of the retaining means.

10. Surgical ring according to claim 9, wherein the first gripping pocket comprises a first cavity designed to contain the filler fluid, said first cavity extending between a bottom wall oriented toward the exterior of said loop and a peak wall oriented toward the interior of said loop, and in that the retaining means is situated at a level between 40% and 60% of a useful height (h₁) of said first cavity measured between the bottom wall and peak wall.

11. Surgical ring according to claim 1, wherein the retaining means, the first gripping pocket and the second gripping pocket demarcate, between the first and second peak portions, a depression for which the ratio of the width at maximum penetration depth to said maximum penetration depth is greater than or equally to 0.35 and preferably appreciably ranging from 0.35 to 1.

12. Surgical ring according to claim 1, wherein the retaining means is preferably made out of a biocompatible elastomer material such as silicone.

13. Surgical ring according to claim 1, wherein the retaining element forms one piece with the first gripping pocket and/or with the second gripping pocket.

14. Surgical ring according to claim 1, wherein it has appreciably a geometry generated by the axial revolution.
